(19) United States
(12) Patent Application Publication
(10) Pub. No.: US 2007/0213628 A1
(43) Pub. Date: Sep. 13, 2007

(54) Respiratory Metabolic Rate Measurement Apparatus

(75) Inventor: Austen Peter Bradley, Padiham (GB)

Correspondence Address: BLACKWELL SANDERS LLP, 720 OLIVE STREET, SUITE 2400, ST. LOUIS, MO, 63101, US

(73) Assignee: NUTREN TECHNOLOGY LIMITED, Padiham (GB)

(21) Appl. No.: 10/598,539

(22) PCT Filed: May 19, 2005
(86) PCT No.: PCT/GB05/01920

§ 371 Date: Oct. 13, 2006

(30) Foreign Application Priority Data
Jun. 16, 2004 (GB) 0413436.7

(51) Int. Cl. A61B 5/083

(52) U.S. Cl. 600/531

(57) ABSTRACT

A (8, 6, 10, 12, 14) metabolic rate measuring apparatus comprising means to measure a reference metabolic rate of a user from at least one breath of the user, means to measure metabolic rate from a subsequent breath of a user, means (14) to indicate a deviation from the reference rate in a breath and prevent measuring of subsequent breaths, and means (50) for a user to override the measurement prevention means to enable measurement of metabolic rate from breaths which deviate from the reference rate.

### FIELD OF THE INVENTION

This invention relates to metabolic rate measurement apparatus and methods of measuring metabolic rate.

### BACKGROUND TO THE INVENTION

Techniques of calorimetry are used to study energy and metabolism in humans and animals. Calorimetry is used, for example, for diagnosis of metabolic disorders and for calculating resting metabolic rate and nutritional requirements of a subject. Calorimetric measurements can be made directly as a measure of heat loss of a subject, or alternatively indirect measurements can be made of a chemical by-product of metabolism. Metabolic rate can be used to determine energy expended by a subject, for example, in Kcal/minute.

Many devices are arranged to measure the resting metabolic rate of a subject, in order to determine calories expended at a resting state, which may help in diagnosis of metabolic disorders, or to monitor fitness of the subject.

In known apparatus for monitoring resting metabolic rate, the apparatus usually contains a reference data which is determined for each subject, and contains data on the average resting metabolic rate of that subject. Reference data is generally inputted over a number of readings by each subject and stored as an average. When the same subject uses the device subsequently, the apparatus checks that the data inputted by the subsequent use of the subject is within the parameters of the reference resting metabolic rate stored in the apparatus. The apparatus can immediately spot a discrepancy and report the deviation. The deviation may be due to a metabolic disorder or illness in the individual, in which case the deviation may be reported to a physician or other health-care professional.

In some instances, it may be desirable for a user to be able to utilise resting metabolic rate apparatus to measure metabolic other than at rest. In the known apparatus, this is not possible due to the referencing of a base metabolic rate, and the indication of an error if a subsequent use of the apparatus by the same subject is outside of the reference parameters.

It would be advantageous to provide a metabolic rate measurement apparatus in which a reference data for resting metabolic rate could be inputted by each user of the apparatus, but in which raised or lowered metabolic rate could be measured as and when required.

It would furthermore be advantageous to provide a metabolic rate measurement rate apparatus in which raised or lowered metabolic rate could be determined by infrequent measurements, enabling a subject to input data when he or she desires, but still benefit from the apparatus calculating the raised or lowered metabolic rate.

It is therefore an aim of preferred embodiments of the invention to overcome or mitigate at least one of the problems of the prior art, whether expressly disclosed herein or not.

### SUMMARY OF THE INVENTION

According to a first aspect of the present invention there is provided a metabolic rate measuring apparatus comprising means to measure a reference metabolic rate of a user from at least one breath of the user, means to measure metabolic rate from a subsequent breath of a user, means to indicate a deviation from the reference rate in a breath and prevent measuring of subsequent breaths, and means for a user to override the measurement prevention means to enable measurement of metabolic rate from breaths which deviate from the reference rate.

The means to measure a reference metabolic rate may comprise an oxygen sensor and/or a carbon dioxide sensor, operatively connected to an electronic calculation means.

The oxygen sensor and/or carbon dioxide sensor may further be connected to a flow meter to measure the volume of inhaled and/or exhaled breaths.

The means to measure a reference metabolic rate may be connected to a data storage means, arranged in use to store the reference metabolic rate data.

The flow meter may comprise any suitable moveable member such as a plunger or paddle wheel.

Suitable oxygen sensors include MOX-1 (RTM) sensors available from City Technology Limited, Portsmouth, UK.

The oxygen sensor and/or carbon dioxide sensor and the flow meter may be arranged to operatively co-operate to measure the reference metabolic rate.

The means to measure the reference metabolic rate may be the same as the means to measure metabolic rate from a subsequent breath of a user. Thus the means to measure metabolic rate from a subsequent breath of a user may comprise an oxygen sensor and/or carbon dioxide sensor, and, optionally, a flow meter, connected to an electronic calculation means.

Suitably the oxygen sensor, carbon dioxide sensor and flow meter, when present, transmit data to the electronic calculation means by means of electronic signals.

Preferably the means to measure the reference metabolic rate comprises means to measure a reference range of metabolic rates. Thus the reference metabolic rate preferably comprises a range of values, preferably in Kcal/min.

The reference metabolic rate is preferably measured as an average from a plurality of breaths, but may be measured from a single breath of a user.

The means to indicate a deviation from the reference metabolic rate may comprise any suitable visual or aural indicator, operatively connected to the means to measure the reference metabolic rate.

Suitable visual indicators include display screens, LED indicators and the like, and the visual indicator may be in the form of text, numerals, warning lights or any mixture thereof.

The means to prevent measurement of subsequent breaths when a deviation from the reference metabolic rate is detected may comprise means to prevent operation of the means to measure subsequent breaths. The measurement prevention means may effect non-recordal of subsequent breaths, until a breath is measured which corresponds to the reference metabolic rate.

The means to prevent measurement of subsequent breaths may effect deactivation of the means to measure subsequent breaths. Where the means to record subsequent breaths comprise an oxygen sensor and/or a carbon dioxide sensor, they may be deactivated by the measurement prevention means.

The means to override the measurement prevention means may comprise means to enable operation of the means to record subsequent breaths.

The means to override the measurement prevention means may comprise an actuator, operable by a user which deactivates the measurement prevention means. The actuator may be connected to a button, lever, touch pad or the like for example, or may be user voice-activated.

The means to override the measurement prevention means thus enables a user to actively enable metabolic rate measurement which deviates from the parameters of the reference metabolic rate, allowing, for example, measurement of metabolic rate during exercise, or to calculate metabolic rate after a defined time period of activity.

The metabolic rate measurement apparatus preferably comprises a housing having a fluid inlet. The housing preferably comprises a fluid outlet. The fluid inlet preferably comprises a mouthpiece, which may be integral with the fluid inlet, or detachable. The mouthpiece may comprise a mask, arranged in use to be secured to a user's mouth.

The metabolic rate measurement apparatus preferably comprises a portable apparatus, more preferably a hand-held apparatus.

The metabolic rate measurement apparatus may be powered by an internal power source such as a power cell or battery, or may comprise means to connect the apparatus to an external power source, such as a mains electricity plug for example.

### BRIEF DESCRIPTION OF THE DRAWINGS

The invention will now be described by way of example only, with reference to the following drawings, in which:

FIG. 1 is a schematic, partially cross-sectional, side view of part of a metabolic rate measuring apparatus in accordance with the present invention, in a start position;

FIG. 2 is a schematic, partially cross-sectional, side view of the apparatus of FIG. 1 in an end position;

FIG. 3 is a schematic, partially cross-sectional, side view of a second embodiment of a metabolic rate measurement apparatus in accordance with the present invention.

### DESCRIPTION OF THE PREFERRED EMBODIMENTS

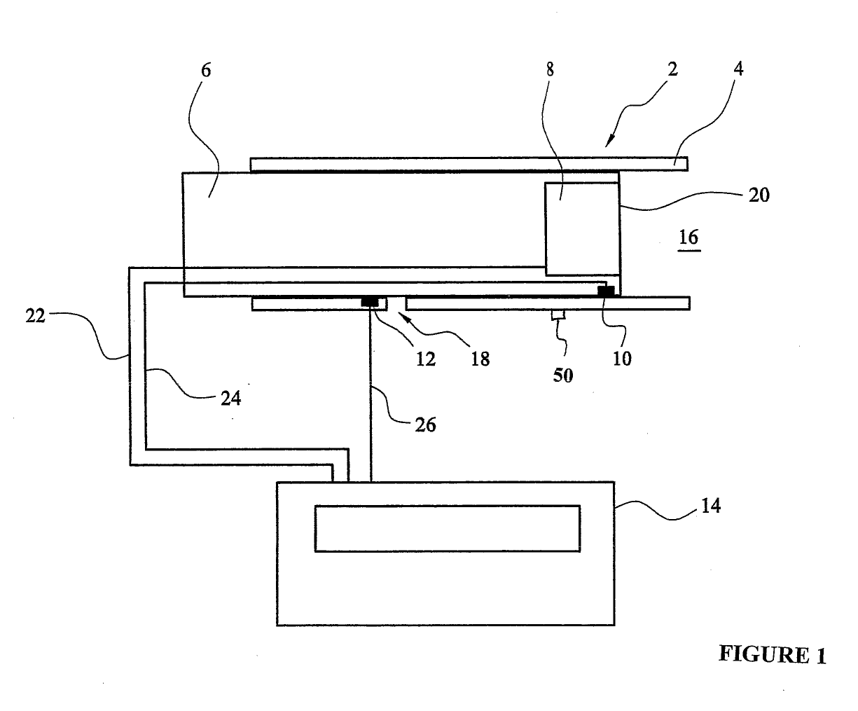

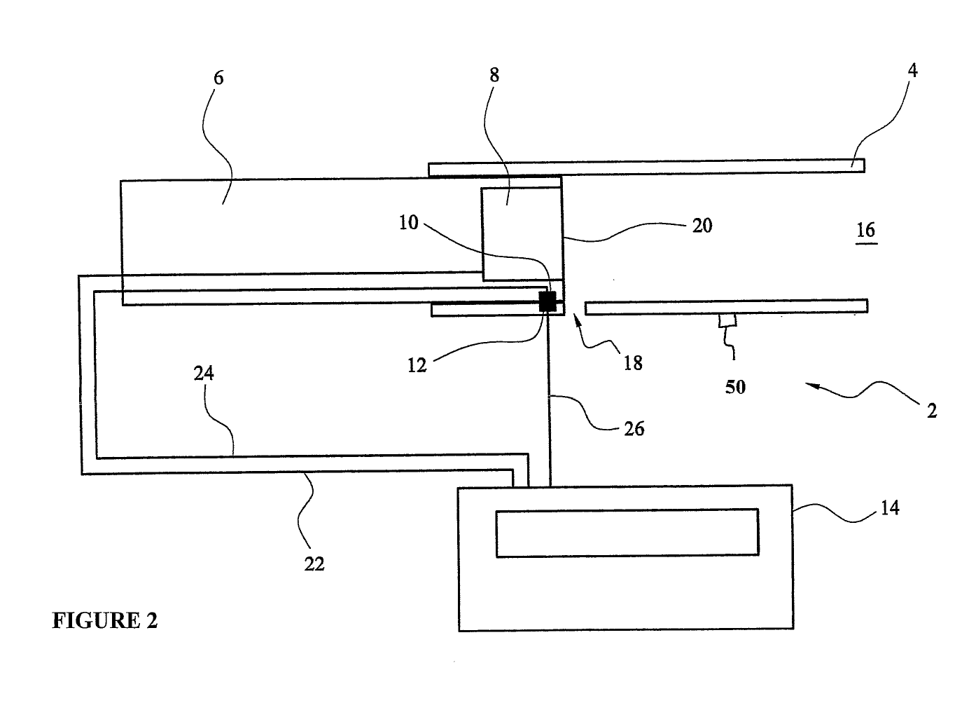

FIGS. 1 and 2 show part of a metabolic rate measurement apparatus of the invention in the form of a calorimeter, suitable for calculating the metabolic rate of a subject. The apparatus 2 comprises a housing 4, means to measure the volume of a user's inhaled breath in the form of a plunger 6 and a movement sensor provided by a first switch 10 and a second switch 12. The apparatus comprises a means to measure the gas content of an exhaled breath in the form of an oxygen sensor 8. The apparatus 2 further comprises electronic calculation means 14. The plunger is slideably moveable inside the housing 4, the interior of the housing 4 and the plunger 6 having the same cross-sectional shape. The relative dimensions of the periphery of the plunger 6 and the interior of the housing 4 being arranged such that there is a substantially fluid-tight fit between the periphery of the plunger 6 and the interior of the housing 4.

The apparatus 2 also includes a means to override the measurement prevention means in the form of an actuator button 50, operably connected to the electronic calculation means 14 by a wire (not shown).

The oxygen sensor 8, the plunger 6 and movement sensor switches 10 and 12, and the electronic calculation means 14 are operably co-operable provide a means to measure a reference metabolic rate from a breath of a user, and metabolic rate from subsequent breaths of a user, based on oxygen content of exhaled breath, in conjunction with the means to measure the volume of breath.

The housing 4 further comprises of a fluid inlet 16 and a fluid outlet 18.

The oxygen sensor 8 is embedded in the end 20 of the plunger 6. The oxygen sensor is electrically connected to the electrical calculation means 14 by wires 22.

The first switch 10 of the movement sensor is embedded in a side of the plunger 4. The second switch 12 of the movement sensor is embedded in the interior wall of the housing 4. A wire 24 connects the first switch 10 with the electrical calculation means 14 and a wire 26 connects the second switch 12 with the electrical calculation means 14.

In use of the apparatus 2, the plunger 6 is arranged in the housing 4 at the start position as shown in FIG. 1. An exhaled breath is guided into the fluid inlet 16 of the housing 4. The end of the housing 4 providing the fluid inlet 16 may act as a mouthpiece for the apparatus 2. Alternatively, a separate mouthpiece (not shown) may be directly or indirectly connected to the housing 4. The exhaled breath causes the plunger 6 to move away from the fluid inlet 16. The plunger 6 will continue to move in this direction until the end 20 of the plunger 6 passes the fluid outlet 18 to reach the end position as shown in FIG. 2. Thereafter, excess exhaled breath will pass out of the fluid outlet 18 and movement of the plunger 6 will cease.

Whilst the plunger 6 is moving from the start position to the end position, the oxygen sensor 8 measures fraction of oxygen in the exhaled breath. The oxygen sensor 8 sends information of the measurement to the electrical calculation means 14 in the form of an electrical signal by means of wire 22. The electrical calculation means calculates the reference metabolic rate of a user based on data received from the movement sensor and oxygen sensor.

The user may then inhale through the fluid inlet 16 in order to move the plunger back from the end position to the start position, or alternatively push the plunger back manually.

In use, the plunger 6, movement switches 10, 12, and the oxygen sensor 8 comprise the means to measure the reference metabolic rate of a user from at least one breath.

In use, a user at rest will exhale into the apparatus 2, in order for the apparatus 2 to measure the reference metabolic rate. The reference metabolic rate may comprise readings from a single exhaled breath, or comprise an average from a plurality of exhaled breaths.

The apparatus 2 is of the size and configuration such that it can be held and carried in the hand by an individual and connected to the electrical calculation means when desired.

Once a reference metabolic rate is established, a user may subsequently breath into the apparatus 2, to measure the metabolic rate from subsequent breaths. The electrical calculation means 14 detect the metabolic rate of the user from subsequent breaths, and compare it to the reference metabolic rate.

If a deviation is detected from the reference metabolic rate, then the device will indicate a deviation, through use of an audio signal from a speaker (not shown), and the electronic calculation means are then arranged to be deactivated to prevent recordal of subsequent breaths.

The electronic calculation means 16 therefore comprises the means to prevent measurement of subsequent breaths.

A user may then override the measurement prevention means by pressing the button 50, which sends a signal to the electronic calculation means 14 to reactivate, and measure subsequent breaths by the user. In this way, breaths which a user knows will be outside of the reference metabolic rate, or believes would be outside of the reference metabolic rate, can then be measured, in order for the apparatus 2 to measure the raised or lowered metabolic rate.

This is particularly useful for users who wish to measure metabolic rate after a certain defined period of activity or exercise.

For example, a user may breath into the apparatus 2 and the electronic calculations means 14 may calculate a reference metabolic rate of 1.5 Kcal/minute. A user may then undertake exercise for 10 minutes, and breath into the device, at which point the electronic calculation means 14, as the means to detect a deviation from the reference metabolic rate, will detect a raised metabolic rate, and prevent recordal of subsequent breaths. The speaker will audibly indicate to the user that a metabolic rate outside of the reference range has been detected, and the electronic calculation means 14 as the measurement prevention means, will deactivate to prevent recordal of subsequent breaths. The user may then press the button 50, to reactivate the electronic calculation means 14, and breath into the device to measure subsequent breaths after exercise. The electronic calculation means 14 can then calculate the raised metabolic rate utilising the plunger 6, movement sensors and oxygen sensor 6, and indicate the value of the raised metabolic rate to the user.

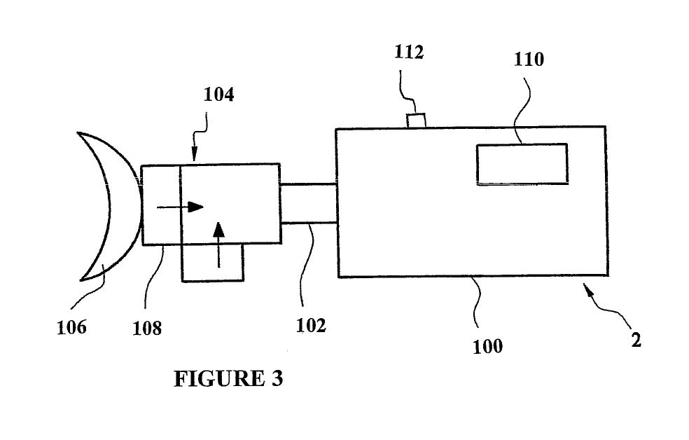

We refer now to FIG. 3 which illustrates the second preferred embodiment of the breath monitoring apparatus of the invention. The breath monitoring apparatus 2 comprises a housing 100 in which is housed a means to measure the volume and gas content of an exhaled breath of a user (not shown). The means to measure the gas content of the exhaled breath comprises a City Technology 40X-1 City Cell Oxygen Meter (supplied by City Technology Ltd, Portsmouth, UK) and the means to measure the volume of the exhaled breath comprises a Honeywell AWM 5104 VN Mask Flow Meter (Honeywell, USA). The housing 100 also includes electronic calculation means (not shown) which comprise the means to calculate the reference metabolic rate of one or more breaths in conjunction with the means to measure the gas content and volume of an exhaled breath of a user. The means to calculate the reference metabolic rate also comprise the means to measure metabolic rate from subsequent breaths of a user.

Extending from one end of the housing 100 is a fluid inlet 102. A detachable mouthpiece 104 is connected to the fluid inlet 102. The mouthpiece 104 includes a T-valve 108, which allows exhaled breath to enter the fluid inlet 102 and into apparatus 2, but allows air from outside the apparatus 2 to be inhaled through the T-valve when a user inhales. The mouthpiece 104 includes the Mask Flow Meter, for measuring the volume of breath of the user. The distal end of the T-valve includes a flexible mask 106, arranged to be connected over the mouth of a user.

The apparatus 2 includes a display screen 110, which acts as a means to indicate a deviation from the reference metabolic rate in a breath. The display screen 110 displays text to a user.

The device includes means for preventing measurement of subsequent breaths upon detection of a deviation from the reference metabolic rate, in the form of the electronic calculation means (not shown)

The apparatus 2 also includes means for user to override measurement prevention means to enable measurement of metabolic rate from breaths which deviate from the reference rate, in the form of an actuator button 112.

Use of the device shown in FIG. 3 is similar to that shown in FIGS. 1 and 2.

In use, a user will exhale into the apparatus 2 and the reference metabolic rate will be calculated by the electronic calculation means, from data supplied by the flow meter and oxygen sensor. The reference metabolic rate may be measured from a single exhalation or an average of a plurality of exhalations. The reference metabolic rate may allow for deviation within a defined range, which may be set by a user or machine set.

Once the reference metabolic rate is determined, a user may then subsequently breathe into the apparatus 2, and the electronic calculations means will measure the metabolic rate of subsequent breaths. If a deviation from the reference rate is detected, then this will be indicated on the display screen 110, and the electronic activation means will deactivate to prevent subsequent breaths from being measured.

The user may override the measurement prevention means by pressing the activator button 112, which sends a signal to the electronic calculation means to reactivate and measure the metabolic rate of subsequent breaths.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extend to any novel one, or any novel combination, of the features disclosed in this specification (including any accompanying claims, abstract and drawings), or to any novel one, or any novel combination, of the steps of any method or process so disclosed.

1. A metabolic rate measuring apparatus comprising means to measure a reference metabolic rate of a user from at least one breath of the user, means to measure metabolic rate from a subsequent breath of a user, means to indicate a deviation from the reference rate in a breath and prevent measuring of subsequent breaths, and means for a user to override the measurement prevention means to enable measurement of metabolic rate from breaths which deviate from the reference rate.

2. An apparatus as claimed in claim 1 wherein the means to measure a reference metabolic rate comprises an oxygen sensor and/or a carbon dioxide sensor, operatively connected to an electronic calculation means.

3. An apparatus as claimed in claim 2 wherein the oxygen sensor and/or carbon dioxide sensor is connected to a flow meter to measure the volume of inhaled and/or exhaled breaths.

4. An apparatus as claimed in claim 1 wherein the means to measure a reference metabolic rate is connected to a data storage means, arranged in use to store the reference metabolic rate data.

5. An apparatus as claimed in claim 1 wherein the means to measure the reference metabolic rate is the same as the means to measure metabolic rate from the subsequent breath of a user.

6. An apparatus as claimed in claim 1 wherein the means to measure the reference metabolic rate comprises means to measure a reference range of metabolic rates.

7. An apparatus as claimed in claim 1 wherein the reference metabolic rate is measured as an average from a plurality of breaths.

8. An apparatus as claimed in claim 1 wherein the means to indicate a deviation from the reference metabolic rate comprises a visual indicator selected from a display screen, an LED, a text display, a numeric display, a warning light or any mixture thereof.

9. An apparatus as claimed in claim 1 wherein the means to prevent measurement of subsequent breaths when a deviation from the reference metabolic rate is detected, comprises means to prevent operation of the means to measure subsequent breaths.

10. An apparatus as claimed in claim 9 wherein the measurement prevention means effects non-recordal of subsequent breaths, until a breath is measured which substantially corresponds to the reference metabolic rate.

11. An apparatus as claimed in claim 1 wherein the means to prevent measurement of subsequent breaths effects de-activation of the means to measure subsequent breaths.

12. An apparatus as claimed in claim 9 when dependent on claim 2, wherein the means to record subsequent breaths comprises an oxygen sensor and/or a carbon dioxide sensor which is de-activated by the measurement prevention means.

13. An apparatus as claimed in claim 1 wherein the means to override the measurement prevention means comprises means to enable operation of the means to record subsequent breaths.

14. An apparatus as claimed in claim 1 wherein the means to override the measurement prevention means comprises an actuator, operable by a user, which de-activates the measurement prevention means when operated by a user.
